(19) United States
(12) Patent Application Publication
(10) Pub. No.: US 2025/0318556 A1
(43) Pub. Date: Oct. 16, 2025

(54) FERMENTATION PROCESS FOR NUTRIENT ENRICHMENT

(71) Applicant: NEW TREAT NUTRITION LTD., Ottawa (CA)

(72) Inventor: Shahrzad RAYATPISHEH, Ottawa (CA)

(73) Assignee: NEW TREAT NUTRITION LTD., Ottawa (CA)

(21) Appl. No.: 19/176,455

(22) Filed: Apr. 11, 2025

Related U.S. Application Data
(60) Provisional application No. 63/633,375, filed on Apr. 12, 2024.

(51) Int. Cl. A23L 11/50; A23L 11/00; A23L 11/30; A61K 31/122; A61K 36/48; C12N 1/20; C12R 1/125

(52) U.S. Cl. CPC A23L 11/50; A23L 11/05; A23L 11/34; A61K 31/122; A61K 36/48; C12N 1/205; C12R 2001/125

(57) ABSTRACT

The present disclosure provides a natural fermentation process to enrich nutrients from a food source. The fermentation process comprises a steeping step, a steaming step, an inoculation step, a controlled fermentation step, a fermentation slowing step, a grinding step, and a drying step.

### CROSS REFERENCE TO RELATED APPLICATION

The present application claims priority to U.S. Provisional Application No. 63/633,375, entitled “FERMENTATION PROCESS FOR NUTRIENT ENRICHMENT” filed on Apr. 12, 2024, the contents of which are incorporated herein by reference in their entirety.

### FIELD

The present disclosure is generally directed to fermentation processes for nutrient enrichment. Specifically, the present disclosure relates to natural and controlled fermentation processes that enrich nutrients from food sources.

### BACKGROUND

The nutritional landscape of modern diets is characterized by a growing dependence on processed and convenient foods, prompting concerns over the decreasing presence of natural nutrients and vitamins in daily intake. Extensive research has underscored the detrimental impacts of this nutritional decline on public health, underscoring the imperative for inventive strategies to counteract this deficit. Moreover, the supplementation of processed foods with artificial vitamins, while a common practice, deviates from the preference for natural, unadulterated sources. There is a pressing need to reverse this trend and to provide a sustainable, natural solution that maintains shelf-stability and convenience for modern lifestyles.

Furthermore, it is widely recognized that vitamins exhibit optimal efficacy when present in their natural complex form, alongside other related nutrients, and in their inherent proportions. This natural synergy enhances absorption and utilization within the body, maximizing the nutritional benefits derived from food consumption. In contrast, the addition of artificial or extracted vitamins to processed foods may lack this intricate balance and can potentially lead to unintended side effects. There is therefore a need for a process to provide natural nutrient complexes, thereby ensuring a harmonious blend of vitamins and nutrients for optimal health outcomes without adverse effects.

Vitamin K plays a pivotal role in human nutrition. Vitamin K exists in two primary forms: vitamin K1 (phylloquinone) and vitamin K2 (menaquinone). While vitamin K1 is abundant in leafy green vegetables and plays a crucial role in blood clotting, vitamin K2 exhibits unique properties essential for bone health, cardiovascular function, and other physiological processes. Studies have shown that a daily adequate intake of vitamin K for adults is around 90-120 micrograms and that a daily intake of over 32.7 micrograms of menaquinone is associated with a lowered risk of coronary heart disease and that a daily intake of 180 micrograms of menaquinone-7 (MK-7) improves osteoporosis.

It is now widely acknowledged that the prevention of osteoporosis must commence during childhood to ensure optimal bone growth and continue throughout life to maintain bone mass. Nutritional factors play a pivotal role in both the development and maintenance of healthy bones. While traditional strategies for preventing osteoporosis have focused primarily on calcium and vitamin D supplementation, recent insights suggest that other nutritional factors may also hold notable importance in bone health.

Vitamin K2 plays a crucial role as a cofactor in enzymatic reactions that govern the activity of osteocalcin, a protein central to the regulation of bone formation. Specifically, its function involves facilitating the carboxylation of osteocalcin, thereby modulating the process of bone mineralization. Beyond its implications for skeletal health, emerging research underscores the significance of vitamin K2 in mitigating the risk of osteoporosis and cardiovascular diseases prevalent in modern societies. Inadequate vitamin K2 intake may contribute to calcium deposition in soft tissues, such as coronary arteries, rather than in bones, exacerbating the risk of arterial calcification and subsequent cardiovascular complications. By promoting proper calcium utilization and ensuring its deposition in bone tissue rather than blood vessel walls, vitamin K2 emerges as a pivotal nutrient in maintaining overall cardiovascular health and reducing the incidence of osteoporosis-related fractures.

In modern diets, and in particular, the Standard American Diet (SAD), there is a notable absence in the current availability of food products, particularly fermented items, that naturally contain vitamin K in its complex form. Such products are essential to fulfill the nutritional requirements of individuals across all age groups, including children, pregnant and breastfeeding women, youth athletes, and the elderly, especially those who may be taking multiple medications and need to avoid potential drug interactions.

Vitamin K2 encompasses several subtypes, with MK-7 being of particular interest due to its long half-life and bioavailability. Despite its significance, obtaining adequate vitamin K2 intake remains a challenge in modern diets, with limited dietary sources available.

Grass-fed and grass-finished animal fat and fermented foods are among the few options, yet uncertainties persist regarding their vitamin K2 content. Traditional lacto-fermentation methods, for instance, often yield insufficient amounts of vitamin K2.

One promising avenue for enriching food products with essential vitamins, particularly vitamin K2, is through fermentation. Fermentation is a metabolic process that involves the conversion of sugars into other compounds by microorganisms like bacteria, yeast, or fungi. Various types of fermentation exist, each facilitated by specific bacterial strains. Utilizing various bacterial strains, fermentation offers a natural method for enhancing the nutritional content of foods. Bacillus subtilis var natto, in particular, is known for its ability to produce high quantities of vitamin K2 during fermentation, with a focus on MK-7 production. However, the challenges faced by the food and supplement industry in incorporating MK-7 from fermentation remain significant.

Natto, a traditional Japanese delicacy comprising fermented soybeans, has gained renown for its rich content of vitamin K2, particularly MK-7, produced during the fermentation process. However, despite its nutritional benefits, the conventional natto fermentation method presents significant challenges. These include intricate procedures and the development of undesirable taste and odor profiles, which have hindered its widespread integration into food production practices.

Synthetically produced menaquinone or menaquinone derived from microbial sources presents its own set of challenges and limitations. While these methods offer a means of obtaining vitamin K2, particularly MK-7, they often involve complex chemical synthesis processes or microbial 1 fermentation techniques. One significant drawback of synthetically produced menaquinone is the potential for impurities and variations in quality, which can arise from the manufacturing process. Moreover, the cost of synthetic production methods can be prohibitive, making it less accessible for widespread use in food fortification or supplementation

Microbial production of menaquinone, particularly MK-7, often involves extraction from bacterial cultures such as Bacillus subtilis. While this method offers a natural source of vitamin K2, it is not without its drawbacks. One significant limitation is the efficiency of the extraction process. Extracting menaquinone from microbial cultures can be complex and labor-intensive, requiring specialized equipment and expertise. Additionally, the yield of menaquinone from microbial cultures may vary, leading to inconsistent production levels and potential supply chain issues. Furthermore, the extraction process may result in the co-extraction of undesirable compounds or impurities, impacting the purity and quality of the final product. These challenges can pose barriers to the scalability and cost-effectiveness of microbial production methods, limiting their feasibility for large-scale commercial applications. Therefore, although microbial production provides a natural source of menaquinone, the drawbacks associated with the extraction method, coupled with the absence of accompanying nutrients that complement MK-7, pose significant challenges.

### SUMMARY

In an aspect, the present disclosure provides a fermentation process for enriching nutrients from at least one food source, the process comprising: steeping the at least one food source to produce a soaked food source; steaming the soaked food source to produce a softened food source; inoculating the softened food source with at least one microorganism to produce an inoculated food source; fermenting the inoculated food source to produce a fermented food source; cooling the fermented food source to slow fermentation; grinding the fermented food source to produce a ground product; and, drying the ground product in a drying step to produce a final product having enriched nutrients.

In another aspect of the present disclosure, the at least one microorganism is at least one of a bacteria and bacterial spores.

In another aspect of the present disclosure, the nutrients comprise vitamin K2.

In yet another aspect of the present disclosure, an amount of vitamin K2 in the final product is controlled by adjusting a temperature and a duration of at least one of the fermenting and drying steps.

In yet another aspect of the present disclosure, an amount of vitamin K2 in the final product is controlled by combining at least two food sources.

In yet another aspect of the present disclosure, an amount of vitamin K2 in the final product is controlled by substituting a first food source from the at least one food source with a second food source.

In yet another aspect, the present disclosure provides a composition comprising the final product of the natural fermentation process.

### BRIEF DESCRIPTION OF THE DRAWINGS

The following figure serves to illustrate various embodiments of features of the disclosure. The figure is illustrative and is not intended to be limiting.

FIG. 1 is a schematic diagram of an overview of a fermentation process for enriching nutrients from a food source, according to an embodiment of the present disclosure.

### DETAILED DESCRIPTION

The following embodiments are merely illustrative and are not intended to be limiting. It will be appreciated that various modifications and/or alterations to the embodiments described herein may be made without departing from the disclosure and any modifications and/or alterations are within the scope of the contemplated disclosure.

The present disclosure provides a fermentation process to enrich nutrients from a food source. Unlike conventional methods reliant on synthetic fortification, the present fermentation process emphasizes sourcing vitamins directly from a food source having an authentic food origin.

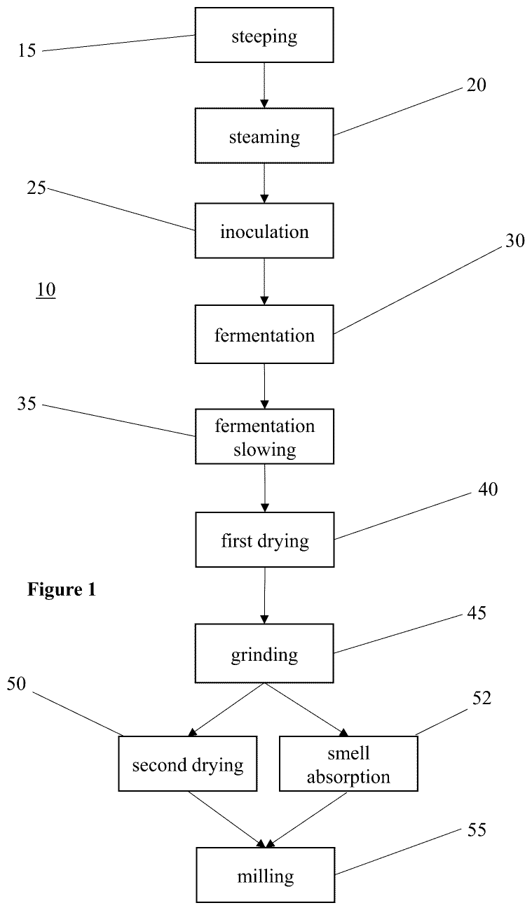

With reference to FIG. 1 and according to an embodiment of the present disclosure, a schematic representation of fermentation process 10 for enriching nutrients from one or more food sources is shown. In a preferred embodiment of the present disclosure, the process 10 generally comprises a steeping step 15, a steaming step 20, an inoculation step 25, a fermentation step 30, a fermentation slowing step 35, a first drying step 40, a grinding step 45, a second drying step 50, and a milling step 55.

In one embodiment of the present disclosure, the steeping step 15, steaming step 20, inoculation step 25, fermentation step 30, and fermentation slowing step 35, can each be performed in separate vessels. In another embodiment of the present disclosure, the aforementioned steps can be performed in a single vessel.

In one embodiment of the present disclosure, the fermentation process 10 can be used to enhance nutrients from one or more food sources using one or more microorganisms. A person of skill in the art will appreciate that using multiple food sources may create unique flavors and nutritional benefits, due in part to using different substrates and microbial activity.

During the steeping step 15, a food source is cleaned and soaked in water, where the food source absorbs water. In one embodiment of the present disclosure, the food source is either a legume or a vegetable, although other food sources can be used such as fruits and dairy. In one embodiment of the present disclosure, the food source is a grain. In one embodiment of the present disclosure, the dairy contains Vitamin D₃. In one embodiment of the present disclosure, the food source is at least one vegetable that is at least one of a starchy root vegetable, a non-starchy root vegetable, and a vegetable from the Cruciferae family. In another embodiment of the present disclosure, the legume is a chickpea. In one embodiment, the legume, such as the aforementioned chickpea, is soaked in water during the steeping step 15 to facilitate sprouting. The duration of sprouting is less than 24 hours. A person of skill in the art will appreciate that steeping and sprouting reduces and breaks down antinutrients, such as lectins. A person of skill in the art will also appreciate that steeping and sprouting increases the nutritional value of the food source.

Upon completion of the steeping step 15, the food source is steamed in the steaming step 20. In one embodiment of the present disclosure, the food source is steamed at a high pressure. In a preferred embodiment of the present disclosure, the high pressure is between 10-12 pounds per square inch (PSI). A person of skill in the art will appreciate that steaming the food source will soften it, reduce the amount of antinutrients therein, and facilitate the subsequent inoculation step 25.

During the inoculation step 25, the softened food source is inoculated at the appropriate temperature with one or more microorganisms to produce an inoculated food source. In one embodiment of the present disclosure, the microorganism is a fungus. In another embodiment of the present disclosure, the microorganism is a bacteria or bacterial spores. In yet another embodiment of the present disclosure, the softened food source may be inoculated with a mixture of fungi and bacteria. In one embodiment of the present disclosure, the bacteria or bacterial spores are derived from Lactobacillus species such as Lactobacillus acidophilus, Lactobacillus plantarum, and Lactobacillus casei. In another embodiment of the present disclosure, the bacteria or bacterial spores are derived from Streptococcus species such as Streptococcus thermophilus. In a preferred embodiment of the present disclosure, the bacteria or bacterial spores are derived from Bacillus subtilis. In another embodiment of the present disclosure, the bacteria or bacterial spores are derived from Bacillus subtilis variant natto.

After the inoculation step 25, the inoculated food source is fermented in fermentation step 30 at a specific temperature, humidity and duration to produce a fermented food source. A person of skill in the art will appreciate that each food source will have its own optimal fermentation temperature and duration. In one embodiment of the present disclosure, the fermentation temperature range is 40-50° C. (104-122° F.), the fermentation duration range is 20-40 hours and the humidity is 70-90%. The duration and temperature of fermentation can be adjusted to alter the vitamin K2 content, the aroma, and the taste of the fermented food source. In one embodiment of the present disclosure, the fermentation step 30 can be divided to have two or more temperature durations. For example, the temperature of the fermentation step 30 may be higher during the first 6 to 10 hours of fermentation while the remaining hours may have a relatively lower temperature.

Fermentation is slowed at fermentation slowing step 35 by cooling the fermented food source for several hours to reduce microorganism activity and to stabilize the fermentation chemical outputs. In one embodiment of the present disclosure, the fermented food source is refrigerated at a temperature of 35-39° F. (1.6-4° C.). In one embodiment of the present disclosure, the fermented food source is refrigerated for 2 to 12 hours. A person of skill in the art will appreciate that the slowing of fermentation may result in beneficial probiotic effects.

After fermentation is slowed, the fermented food source enters a first drying step 40, where the fermented food source is dried at a temperature that is the same as the fermentation temperature at fermentation step 30. To preserve vitamin content, the duration of the first drying step 40 is reduced. In a preferred embodiment of the present disclosure, the duration of the first drying step 40 is 1-3 hours. Light exposure is reduced during the first drying step 40 to conserve vitamin content. A person of skill in the art will appreciate that the first drying step 40 is beneficial for decreasing any sliminess that may be found in the fermented food source. First drying step 40 also produces a dried product that will be easier to grind during grinding step 45.

The dried product is ground during the grinding step 45 to decrease the particle size of the dried product. In one embodiment of the present disclosure, the grinding step 45 is brief and is done in the absence of light. The decreased particle size allows for easier drying in the subsequent second drying step 50. In a preferred embodiment of the present disclosure, the particle size of the ground product is less than 1 mm in diameter.

The ground product is dried a second time during second drying step 50. As was the case during the first drying step 40, the ground product is dried at the same temperature as the fermentation step 30. A person of skill in the art will appreciate that the temperature during the second drying step 50 plays a crucial role in preserving not just vitamin K2, but also other enzymes and nutrients. A person of skill in the art will also appreciate that the second drying step 50 facilitates the subsequent milling step 55. The ground product is dried until dehydration is complete. In one embodiment of the present disclosure, dehydration is complete when the ground product has less than 5% moisture content. In an optional step of the present disclosure, unwanted smells from the ground product are absorbed using one or more natural products during a smell absorption step 52 that may occur in parallel to the second drying step 50. The natural products are placed in proximity of the ground product. In an embodiment of the present disclosure, the natural products do not contact the ground product. In yet another embodiment of the present disclosure, the natural products are charcoal and sodium bicarbonate or a combination of the two.

The dehydrated ground product is milled during milling step 55 to produce a final product. In one embodiment of the present disclosure, the final product is milled to a powder for easier application to drink items such as smoothies. In another embodiment of the present disclosure, the final product is milled to a coarse texture for application to food items such as meatloaves. In one embodiment of the present disclosure, the powdered final product is subsequently packaged in dark packaging to avoid destruction of vitamin K2 by light exposure.

The present fermentation process 10 allows the controlled enrichment of vitamins in the final product by adjusting the temperature and duration of the fermentation step 30, first drying step 40, and second drying step 50. In contrast, conventional methods do not provide a mechanism to control vitamin yield and instead aim for the highest production of vitamins through extraction. In another embodiment of the present disclosure, the production of vitamin K2 can be controlled during the fermentation process 10 by controlling the vitamin K1 content of the food source. In another embodiment of the present disclosure, the production of vitamin K2, and in particular MK-7, can be controlled by controlling the duration and temperature of the fermentation step 30.

In one embodiment of the present disclosure, the first drying step 40 and the second drying step 50 can be replaced with a single drying step. In a preferred embodiment of the present disclosure, the single drying step can be a freeze-drying step.

Additionally, the present fermentation process 10 does not discard the food source during the process. The present fermentation process 10 is therefore more environmentally friendly than conventional processes and the final product has more nutrients, such as proteins.

In one embodiment of the present disclosure where chickpeas are used as the food source for the present fermentation process 10, the amount of vitamin K2 in the chickpeas after dehydration was equal to or higher than the amount of vitamin K2 in the chickpeas prior to dehydration, thereby indicating a 100% retention of vitamin K2 in the powdered final product. Additionally, the present fermentation process 10 can be controlled according to the embodiments described in the present disclosure such that the amount of vitamin K2 in the final product can be adjusted from 10 micrograms of vitamin K2 per 100 grams of food source to 1000 mcg of vitamin K2 per 100 grams of food source. The amount of vitamin K2 in the final product can be controlled by adjusting the food source, the temperatures, and the durations of the fermentation step, the first drying step, and the second drying step.

The present fermentation process 10 enriches a food source with vitamin K2 in its natural form without extraction and as such, offers several advantages. Firstly, natural sources of vitamin K2, such as fermented foods, contain a complex array of nutrients and cofactors that work synergistically to enhance its absorption and utilization in the body. These complementary compounds, often absent in extracted forms, contribute to the overall health benefits derived from vitamin K2 consumption.

In one embodiment of the present disclosure, the complex array of nutrients includes a range of enzymes, including proteases, amylases, lipases, and cellulases, which help break down proteins, carbohydrates, fats, and fibers in the food source. These enzymes can enhance nutrient availability and digestion in fermented foods.

In another embodiment of the present disclosure, the complex array of nutrients includes vitamins in addition to vitamin K2, such as vitamin B12, riboflavin (vitamin B2), and folate (vitamin B9). Additionally, Bacillus subtilis fermentation may enhance the bioavailability of other vitamins present in the food source.

In another embodiment of the present disclosure, the complex array of nutrients includes other forms of menaquinone, such as MK-4, MK-5, MK-6, MK-8, MK-9, and MK-10.

In another embodiment of the present disclosure, the present fermentation process 10 increases the concentration of smaller peptides and free amino acids in the food source. A person of skill in the art will appreciate that the amino acids can serve as the building blocks of proteins and that they play essential roles in various physiological functions of the body.

In one embodiment of the present disclosure, when Bacillus subtilis is used during the fermentation process 10, short-chain fatty acids (SCFAs), such as acetate, propionate, and butyrate, are produced through the fermentation of dietary fibers and other carbohydrates present in the food source. A person of skill in the art will appreciate that SCFAs serve as an energy source for intestinal cells and help maintain gut health. Additionally, Bacillus subtilis produces antimicrobial compounds, such as bacteriocins and organic acids, which inhibit the growth of harmful bacteria and fungi in the food source.

Additionally, natural sources of vitamin K2 are less likely to contain impurities or artificial additives that may be present in extracted forms. Furthermore, the natural form of vitamin K2 is more readily recognized and utilized by the body, potentially leading to greater efficacy and bioavailability. Additionally, consuming vitamin K2 in its natural form aligns with the principles of holistic nutrition that emphasizes whole foods with minimal processing for optimal health outcomes. Finally, the controllability of the present fermentation process 10 allows a consumer to know how much vitamin K2 is in their food, which ensures a reliable outcome and prevents overdose.

In one embodiment of the present disclosure, the final product of the fermentation process 10 can be used to naturally enrich food items. In one embodiment of the present disclosure, the final product is shelf stable. By integrating the final product into diverse food items, the principles of authenticity and purity are upheld, thereby presenting a holistic solution for enhancing nutritional value while preserving the integrity of the food.

In another embodiment of the present disclosure, the final product can be used to maintain a user's health and wellbeing. In another embodiment of the present disclosure, the final product can be used to prevent or treat a variety of diseases, including cardiovascular diseases and osteoporosis.

In one embodiment of the present disclosure, the final product of the present fermentation process 10 can be included in natural nutraceutical flakes or powders designed to support health and well-being including, but not limited to, stronger teeth and bones, improved muscle quality, promoting beneficial gut bacteria, anti oxidation, immunomodulation, increasing energy, and delaying aging.

In one embodiment of the present disclosure, the final product of the present fermentation process 10 can be included in natural nutraceutical flakes or powders designed to prevent non-communicable diseases including, but not limited to, cardiovascular, osteoporosis, tooth decay, diabetes, obesity, and multiple cancers.

In one embodiment of the present disclosure, the final product of the present fermentation process 10 can be included in nutraceutical powders to be integrated in other commercial food sources including but not limited to dairy and meat substitutes.

In another embodiment of the present disclosure, the final product contains 180 micrograms of MK-7 per 100 grams of final product. The final product may be used as a natural supplement for athletes.

Many modifications of the embodiments described herein as well as other embodiments may be evident to a person skilled in the art having the benefit of the teachings presented in the foregoing description and associated drawings. It is understood that these modifications and additional embodiments are captured within the scope of the contemplated disclosure which is not to be limited to the specific embodiment disclosed.

1. A fermentation process for enriching nutrients from at least one food source, the process comprising:
steeping the at least one food source to produce a soaked food source;
steaming the soaked food source to produce a softened food source;
inoculating the softened food source with at least one microorganism to produce an inoculated food source;
fermenting the inoculated food source to produce a fermented food source;
cooling the fermented food source to slow fermentation;
grinding the fermented food source to produce a ground product; and,
drying the ground product in a drying step to produce a final product having enriched nutrients.

2. The fermentation process of claim 1, wherein the at least one microorganism is at least one of a bacteria and bacterial spores.

3. The fermentation process of claim 1, wherein the nutrients comprise vitamin K2.

4. The fermentation process of claim 3, wherein an amount of vitamin K2 in the final product is controlled by adjusting a temperature and a duration of at least one of the fermenting and drying steps.

5. The fermentation process of claim 3, wherein an amount of the vitamin K2 in the final product is controlled by combining at least two food sources.

6. The fermentation process of claim 3, wherein an amount of the vitamin K2 in the final product is controlled by substituting a first food source from the at least one food source with a second food source.

7. The fermentation process of claim 1, wherein the at least one food source is selected from at least one of a legume, a grain, and a vegetable.

8. The fermentation process of claim 7, wherein the at least one food source is a chickpea.

9. The fermentation process of claim 1, wherein the soaked food source is steamed at a pressure range of 10 to 12 PSI.

10. The fermentation process of claim 2, wherein the at least one microorganism is Bacillus subtilis.

11. The fermentation process of claim 10, wherein the at least one microorganism is Bacillus subtilis var natto.

12. The fermentation process of claim 1, wherein a duration range of the fermentation step is from 20 to 40 hours and a temperature range of the fermentation step is 40° C. to 50° C.

13. The fermentation process of claim 1, wherein the fermented food source is refrigerated at a temperature range of 1.6° C. to 4° C. for 2 hours to 12 hours during the cooling step.

14. The fermentation process of claim 1, wherein the ground product from the grinding step is less than 1 mm in diameter.

15. The fermentation process of claim 1, wherein the ground product is dried until the ground product has less than 5% moisture content.

16. The fermentation process of claim 1, further comprising an additional drying step between the cooling step and the grinding step.

17. The fermentation process of claim 16, wherein a duration range of the additional drying step is 1 hour to 3 hours.

18. The fermentation process of claim 1, wherein a smell absorption step occurs in parallel with the drying step to reduce unwanted smells from the final product.

19. The fermentation process of claim 1, wherein the final product is milled to produce a powdered product.

20. The fermentation process of claim 19, wherein the powdered product is packaged in dark packaging to protect vitamin K2 from light exposure.
